(19) United States
(12) Patent Application Publication
(10) Pub. No.: US 2002/0193334 A1
(43) Pub. Date: Dec. 19, 2002

(54) Cell-free assay for plant gene targeting and conversion

(76) Inventors: Gregory D. May, Ardmore, OK (US); Eric B. Kmiec, Landenberg, PA (US); Michael C. Rice, Newtown, PA (US)

Correspondence Address: Daniel Becker, Esq., FISH & NEAVE, 1251 Avenue of the Americas, New York, NY, 10020-1104, US

(21) Appl. No.: 10/082,476

(22) Filed: Feb. 20, 2002

Related U.S. Application Data
(63) Continuation of application No. PCT/US00/22974, filed on Aug. 21, 2000.
(60) Provisional application No. 60/149,987, filed on Aug. 20, 1999.

(51) Int. Cl. A61K 48/00; C12Q 1/68; C12N 15/00; C12N 15/87

(52) U.S. Cl. 514/44; 435/455; 435/6; 435/320.1

(57) ABSTRACT

The invention is based on the reaction of oligonucleotides in a cell-free system containing a plant cell extract and a test duplex DNA on a plasmid. The reaction specifically corrects or causes a mutation in a selectable marker gene to a form that can be selected in transformed MutS and RecA deficient bacteria. After transformation into MutS and RecA deficient bacteria, the gene conversion can be detected and quantified.

### TECHNICAL FIELD OF THE INVENTION

The invention relates to genetic modification in plants.

### BACKGROUND OF THE INVENTION

Gene targeting and conversion by site-directed mutagenesis can be useful in accomplishing genetic modification of cells without the integration of foreign DNA into the genome. To this end, chimeric RNA/DNA oligonucleotides have been reported to direct single base changes in episomal and chromosomal targets in mammalian cells (Yoon, et al. 1996. “Targeted gene correction in mammalian cells mediated by a chimeric RNA/DNA oligonucleotide,” Proc Natl Acad Sci USA 93:2071-2076; Cole-Strauss, et al. 1996. “Correction of the mutation responsible for sickle cell anemia directed by a chimeric RNA/DNA oligonucleotide,” Science 273:1386-1389; Kren, et al. 1998. “In vivo site-directed mutagenesis of the factor IX gene by chimeric RNA/DNA oligonucleotides,” Nature Med 4:1-6; Alexeev, V. and Yoon, K. 1998. “Stable and inheritable changes in genotype and phenotype of albino melanocytes induced by an RNA-DNA oligonucleotide,” Nature Biotech 16:1343-1346; and Lai, L. -W. and Lien, Y. -H. H. 1999. “Homologous recombination-based gene therapy,” Exp Neph 7:11-14). The process by which these nucleotide conversions are made is still undefined, but recent evidence suggests that mismatch repair plays a critical role in mammalian cells. Using cell-free extracts from human HuH7 cells, Kmiec and colleagues (Cole-Strauss, et al. 1999. “A mammalian cell-free extract that directs chimeric RNA/DNA oligonucleotide-mediated gene targeting,” Nucl Acids Res 27:1323-1330) demonstrated that both point mutations and frameshift mutations can be corrected by chimeric oligonucleotides and that the reaction is reduced significantly in extracts that lack a functional mismatch repair system. In addition, antibodies directed against hmsh2, the human homolog of the MutS protein from Escherichia coli, are deficient in promoting the chimera-based reaction.

Gene targeting and conversion by site-directed mutagenesis also provide significant advantages in applications to plant systems. For example, chimeric RNA/DNA oligonucleotide-directed gene conversion could be used in: (1) restoration of disrupted genes in plants or the deliberate inactivation of specific plant genes to improve crop value, (2) the ability to create targeted gene modifications to enable the study of gene function, and (3) the production of novel traits in plants without introducing foreign genes.

However, there is a paucity of experimental evidence for defining chimeric RNA/DNA oligonucleotide-directed gene conversion reactions in plant cells (Britt, A. B. 1996. “DNA damage and repair in plants,” Ann Rev Plant Physiol Plant Mol Biol 45:75-100), despite the fact that DNA repair processes impact broad areas of basic and applied plant research including the control of cell cycle and specificity, or lack thereof. In their commentary on gene therapy in plants, Hohn and Puchta pointed out that specific chimeric RNA/DNA oligonucleotides had been used to induce point mutations in several mammalian genes and that chimeric oligonucleotide-dependent mismatch DNA repair had been used in plants (tobacco and maize). A tobacco tissue culture line, a cultured maize line, and immature maize embryos have been treated with chimeric oligonucleotides using microparticle bombardment. Delivery of the chimeric oligonucleotide to the plant cells was reported to be difficult due to the relatively rigid plant cell wall, resulting in low transformation frequencies. Moreover, inconsistent genetic alteration of the plant cell DNA was noted. With the tobacco cell line, DNA repair was shifted from the expected second position of the target codon to the first position. Likewise, in maize, the target codon as well as the codon 5 ′ to it was changed. Thus, significant problems were identified in the chimeric RNA/DNA oligonucleotide-directed gene conversion of plant cells, including low frequency of gene conversion and conversion at non-target nucleotide positions. (Hohn, B. and Puchta, H. 1999. “Gene therapy in plants,” Proc Natl Acad Sci USA 96:8321-8323).

In exemplary studies, Zhu et al. and Beetham et al. reported the first uses of chimeric oligonucleotides to cause site-specific base changes in plants and plant cells (Zhu, et al. 1999. “Targeted manipulation of maize genes in vivo using chimeric RNA/DNA oligonucleotides,” Proc Natl Acad Sci USA 96:8768-8773; and Beetham, et al. 1999. “A tool for functional plant genomics: chimeric RNA/DNA oligonucleotides cause in vivo gene-specific mutations,” Proc Natl Acad Sci USA 96:8774-8778). In these studies, chimeric RNA/DNA oligonucleotide molecules were used to mediate single base changes in plant cells. Zhu et al. reported site-specific heritable GFP mutations in maize genes engineered by introducing chimeric RNA/DNA oligonucleotides into cultured maize cells as well as immature embryos via particle bombardment. Beetham et al. carried out similar studies utilizing electroporation and particle bombardment to deliver chimeric RNA/DNA oligonucleotides to tobacco Nt-1 cells, thereby conferring herbicide resistance in the tobacco cells.

The utility of oligonucleotide-directed mutagenesis in plants as performed by Zhu et al. and Beetham et al. is limited by low gene conversion frequencies which makes screening after gene conversion highly labor and time intensive. In the study of Zhu et al. (Zhu, et al. 1999. Proc Natl Acad Sci USA 96:8768-8773), the reported frequency of site-specific targeting was much less than the frequencies found for chimeric RNA/DNA oligonucleotide repairs in mammalian cells, although the frequency in plants was higher than the frequencies of spontaneous mutation and gene targeting by homologous recombination. Moreover, while the predicted DNA change was obtained in about 85% of the clones, alternative mutations occurred in adjacent bases. In the system taught by Beetham et al, the site of the observed modified base was always found to be in the targeted codon, however, it was shifted one nucleotide 5′ of the target mismatched nucleotides, and frequencies of gene conversion were low.

As pointed out by Hohn and Puchta, plant cells do not provide a controlled biochemical environment in which to study genes and/or proteins associated with gene conversion, and methods utilizing plant cells do not provide optimum conditions under which observed shifting of DNA repair from the targeted positions can be studied. Inherent experimental difficulties encountered with cell-based systems include but are not limited to (1) the frequency of successful particle bombardment or electroporation of plant cells being limited by the rigid plant cell wall, (2) methods for detecting transformation in tissue such as germ tissue or embryos being difficult and time-consuming, and (3) inconclusive analysis due to differing transformation results obtained with varying target plant tissue.

Alternatives to cell-based systems have been reported as providing genetically tractable system for the study and improvement of gene targeting in non-plant systems. Hotta, et al. 1985. Chromasoma 93:140-151; Kucherlapati, et al. 1985. Mol Cell Biol 5:714-720; Jessberger, R. and Berg, P. 1991. Mol Cell Biol 11:445; and Lopez, et al. 1992. Nucleic Acids Res 20:501-506 disclose methods for cell-free homologous recombination.

Reports of mismatch repair in cell-free non-plant systems can also be found in Muster-Nassal and Kolodner. 1986. Proc Natl Acad Sci USA 83:7618-7622 (yeast); Glazer, et al. 1987. Mol Cell Biol 7:218-224 (HeLa cell); Thomas, et al. 1991. J Biol Chem 266:3744-3751 (HeLa cell); Holmes, et al. 1991. Proc Natl Acad Sci USA 87:5837-5841(HeLa cell and Drosophila) and Lahue, et al. 1989. “DNA mismatch correction in a defined system,” Science 245:160-164. The HeLa and Drosophila cell-free systems required that one strand of the mismatched duplex be nicked for full activity. By contrast, reports of repair in Xenopus egg extracts did not require that the mismatched duplex be nicked (Varlet, et al. 1990. Proc Natl Acad Sci USA 87:7883-7887). However, in Varlet, the mismatch was repaired in a random fashion, i.e., the strands acted as templates with equal frequency. Many of the genes required for mismatch repair in yeast and humans have been cloned based on homology with the E. coli mismatch repair genes (Kolodner, R. 1996. Genes Develop 10:1433-1442). Cells having defective mismatch repair genes show genetic instability, termed Replication Error (RER), particularly evident in microsatellite DNA, and malignant transformation. Extracts of RER cells do not have mismatch repair activity (Umar, et al. 1994. J Biol Chem 269:14367-14370). Mismatched repair systems in bacteria are also presented in U.S. Pat. No. 6,004,804 and International Patent Application WO 9958723 for Kumar et al. These studies indicated that successful mismatch repair depends on many variables and that information learned from one system does not necessarily apply to other systems.

To provide for a genetically tractable system for the study and improvement of gene targeting in plants, a cell-free assay system utilizing cell-free extracts in recombinagenic oligonucleotide gene conversion has now been found.

### SUMMARY OF THE INVENTION

In one aspect, the present invention is a method of modifying a target site of a gene-of-interest comprising (1) providing an oligonucleotide that encodes a modification of a gene-of-interest, a duplex DNA molecule containing the gene-of-interest operably linked to a promoter so that the gene of interest can be expressed in a host organism, a cell-free enzyme mixture comprising recombination and gene repair activities and a mismatch repair activity; (2) reacting the oligonucleotide, the duplex DNA molecule, and the cell-free enzyme mixture whereupon the gene-of-interest is modified at the target site to form a modified gene of interest; (3) introducing the modified gene-of-interest into the host organism; and (4) detecting the expression of the modified gene-of-interest. The oligonucleotide can comprise at least 20 and less than or equal to 200 nucleotides. The oligonucleotide can comprise at least 10 and less than or equal to 100 Watson-Crick nucleotide pairs. The oligonucleotide can comprise a single 3′ end and a single 5′ end. In this method, the expression of the modified gene-of-interest can confer a selectable trait on the organism. Alternatively, expression of the modified gene-of-interest can confer an observable trait on the organism.

In another aspect, the present invention in a method of altering a DNA sequence comprising (1) providing an oligonucleotide that encodes a modification of a DNA sequence, a duplex DNA molecule containing the DNA sequence, and a cell-free enzyme mixture comprising recombination and gene repair activities and a mismatch repair activity, (2) reacting the oligonucleotide, the duplex DNA molecule and the cell-free enzyme mixture comprising recombination and gene repair activities and a mismatch repair activity, whereupon the DNA sequence is modified to form an altered DNA sequence, and (3) detecting the altered DNA sequence. The method can further comprise fractionating a cell-free composition so as to enrich the altered DNA sequence relative to the DNA sequence prior to detecting the altered DNA sequence. The oligonucleotide can comprise at least 20 and less than or equal to 200 nucleotides. The oligonucleotide can comprise at least 10 and less than or equal to 100 Watson-Crick nucleotide pairs. The oligonucleotide can comprise a single 3′ end and a single 5′ end. The oligonucleotide can be a duplex mutational vector comprising a contiguous single-stranded self-complementary oligonucleotide having a 3′ end and a 5′ end, wherein the 3′ end and the 5′ end are juxtaposed and wherein at least five contiguous nucleotides are Watson-Crick base paired, the sequence of the oligonucleotide comprising a template for the modified DNA sequence.

In yet another aspect, the present invention is a cell-free composition for the modification of a DNA sequence comprising a duplex DNA containing a target sequence, an oligonucleotide which targets the DNA sequence and encodes the modification thereof, a cell-free enzyme mixture comprising recombination and gene repair activities, and a reaction buffer. In the composition, the oligonucleotide can comprise at least 20 and less than or equal to 200 nucleotides. The oligonucleotide can comprise at least 10 and less than or equal to 100 Watson-Crick nucleotide pairs. The oligonucleotide can comprise a single 3′ and a single 5′ end. The duplex DNA sequence can be a portion of a gene-of-interest that is operably linked to a promoter, so that the gene-of-interest can be expressed in a host organism. The cell-free enzyme mixture can lack mismatch repair activity, with the recombination and gene repair activities being provided by a eukaryote-derived enzyme. Alternatively, the cell-free enzyme mixture can comprise recombination and gene repair activities and further comprise mismatch repair activity being provided by a eukaryote-derived enzyme. The cell-free enzyme mixture can be a defined enzyme mixture of purified plant, yeast or mammalian recombination and repair proteins capable of catalyzing gene repair. The cell-free enzyme mixture can be an extract of a eukaryotic cell such as a plant cell. In the composition, the oligonucleotide can be a duplex mutational vector comprising a contiguous single-stranded self-complementary oligonucleotide having a 3′ end and a 5′ end, wherein the 3′ end and the 5′ end are juxtaposed and wherein at least five contiguous nucleotides are Watson-Crick base paired, the sequence of the oligonucleotide comprising a template for the modified DNA sequence.

### BRIEF DESCRIPTION OF THE DRAWINGS

FIGS. 1A and 1D depicts targeted plasmid sequences and chimeras designed to repair indicated mutations. The plasmids displayed above contain mutations at the indicated sites: plasmid pK^{S}m4021 at base 4021 (FIG. 1A) (SEQ ID NO: 1; SEQ ID NO:2, wild-type; SEQ ID NO:3, mutant; SEQ ID NO:4, converted); plasmid pT^{S}m153 at base 153 (FIG. 1B) (SEQ ID NO:5; SEQ ID NO:6, wild-type; SEQ ID NO:7, mutant; SEQ ID NO:8, converted); and plasmid pT^{S}Δ208 at base 208 (FIG. 1C) (SEQ ID NO:9; SEQ ID NO:10, wild-type; SEQ ID NO: 11, mutant; SEQ ID NO:12, converted). These mutations are in the coding regions of genes that confer antibiotic resistance. Plasmids pK^{S}m4021 and pT^{S}m153 contain point mutations, while pT^{S}Δ208 harbors a frameshift mutation. The sequence of the wild-type, mutant and converted bases are listed below the specific chimeric oligonucleotide designed to correct the mutation. DNA bases are presented in upper case, while RNA nucleotides are presented in lower case. Targeted bases are indicated by an arrow. The chimeric oligonucleotide SC1 (FIG. 1D) (SEQ ID NO: 13), is a non-specific control bearing no sequence homology to any of the gene targets.

FIG. 2 depicts a model for chimera-directed gene repair. The RNA portions of the chimeric oligonucleotide is represented by broken lines. The chimeric oligonucleotide interacts with the DNA target in a process known as homologous recombination forming a double D-loop juncture. The joint molecule complex is recognized by the cell's DNA repair machinery. The mis-paired bases are corrected through the activity of the mismatch repair pathway. The overall process is envisioned as a concerted series of steps using enzymes involved in the process of homologous pairing for the pairing phase, followed by the repairing phase, which is dependent at least on the protein, msh2, or its analogs.

FIG. 3 depicts the correction of the 4021 kan mutation. The targeted plasmid and sequence are displayed (SEQ ID NO: 1, wild-type; SEQ ID NO:2, mutant; SEQ ID NO:3, converted), as well as the DNA sequence of the resulting clones exhibiting resistance to kanamycin (Kan4021(−), SEQ ID NO:2; Kan4021 C Maize, SEQ ID NO: 14; Kan4021C Musa, SEQ ID NO: 15; Kan4021C Tobacco, SEQ ID NO: 16; and Kan4021C Tobacco, 4021 mix, SEQ ID NO: 17). The indicated extract is listed in the left side of the panel, and the altered base from the coding strand of the target is positioned vertically down the page. Without treatment, the G residue is observed. The chimeric oligonucleotide used in the reaction is listed with the source of the cell-free extract. The term “4021 mix” indicates the presence of a multiple base readout at the target site, in this case printed as an “N” within the sequence.

FIG. 4 depicts the correction of the 153 tet mutation. Plasmid and chimera are listed as described in the legend to FIG. 3 except that the target was the tet mutation with an A residue designated for alteration. The left panel represents the sequence data from colonies exhibiting resistance to tetracycline (wild type, SEQ ID NO:6; mutant, SEQ ID NO:7; converted, SEQ ID NO:8; Tet153(−), SEQ ID NO:7; Tet153T Maize, SEQ ID NO:18; Tet153T Musa, SEQ ID NO:19; Tet153T Tobacco, SEQ ID NO:20; Tet153T Tobacco, 153 mix, SEQ ID NO:21). The right panel displays the sequence of the silent marker base created within this plasmid to identify the correct plasmid constructs (wild type, SEQ ID NO:22; mutation, SEQ ID NO:23; Tet153(−), SEQ ID NO:23; Tet153T Maise, SEQ ID NO:24; Tet153T Musa, SEQ ID NO:25; Tet153T Tobacco, SEQ ID NO:26; Tet153T Tobacco, 153 mix, SEQ ID NO:27. The specific chimeric oligonucleotide used in the reaction is listed with the source of the cell-free extract. The term “153 mix” refers to the presence of multiple peaks appearing in the sequence at the target site.

FIG. 5 depicts the correction of the 208 tet mutation. Plasmid pT^{S}Δ208 contains a frameshift mutation at nucleotide position 208 (note triangular marker in mutant sequence listing). Sequence data from resistant colonies resulting from treatment with the indicated cell-free extract are displayed with the targeted site of the inserted base C (wild-type, SEQ ID NO: 10; mutant, SEQ ID NO: 11; converted, SEQ ID NO: 12; TetΔ208(−), SEQ ID NO: 11; Tet208C Maize, SEQ ID NO:28; Tet208C Musa, SEQ ID NO:29; Tet208C Tobacco, SEQ ID NO:30; Tet208C Tobacco, 208 mix, SEQ ID NO:31; and Tet208C Tobacco, 214 insertion, SEQ ID NO:32). The term “208 mix” refers to the presence of multiple peaks appearing in the sequence at the site of insertion, here depicted by an “N”. In the last panel, nucleotide position 214, the next to the last base, depicts the non-specific insertion of a C residue at that site.

### DETAILED DESCRIPTION

We have now found a cell-free assay for oligonucleotide-directed mutagenesis in plant systems. In general, the cell-free assay consists of (1) an in vitro reaction involving an oligonucleotide of interest, a DNA target, a reaction buffer, and a cell-free extract taken from plant cells of interest and (2) a bacterial detection system for determining gene conversion. In the cell-free assay, gene conversion is conducted in a biochemically controlled environment within a genetically tractable system. The robustness of the system allows, for example, direct comparison of various oligonucleotides, targeting of various types of gene conversions, including point mutations or frameshift mutations, and exploration of various components of plant cell extracts taken from various plant cells. Furthermore, the cell-free assay is useful for elucidating certain DNA repair pathways in plant cells as well as the identification and characterization of proteins involved in DNA repair, or other processes. Cell-free extracts from monocotyledonous and dicotyledonous plant species as well as embryonic tissue can be used in conjunction with an oligonucleotide to direct gene conversions

In a preferred embodiment, the cell-free assay provides a method by which a cell-free extract from a plant of interest is screened for its ability to support point mutation or frameshift mutation gene conversion. The demonstration that the cell-free extract supports the correction of a point mutation and/or frameshift mutation indicates that the source plant cells or tissue possess the machinery to catalyze correction of either one or both types of mutations. Accordingly, an objective of the present invention is an assay that can use DNA targets and oligonucleotides to rapidly evaluate the compatibility between different types of oligonucleotides and the recombination and repair enzymes of different phyla, e.g., determining whether the recombination and mismatch repair enzymes of bacteria, plants, insects and mammals have differing substrate preferences.

A further objective of the invention is an assay that can rapidly determine whether a tissue or cell line is a viable target for gene targeting through oligonucleotide induced gene conversion, i.e., whether it contains the requisite enzymes. A yet further objective is an assay to determine what agents or treatments can alter the level of gene targeting activity in a cell line or tissue. A yet further objective of the invention is an assay that can determine whether a compound is an agonist or antagonist of the recombination and repair pathway. An additional objective of the invention is a practical method of making specific genetic changes in a DNA sequence in a cell-free system that is an alternative to polymerase chain reaction PCR-based methods.

The present invention meets these objectives by the discovery that gene targeting can be performed in a cell-free system in plants. The components of the cell-free system are an enzyme mixture containing pairing activity and a mismatch repair activity, a target DNA sequence, and an oligonucleotide. The enzyme mixture can be made by obtaining a cell extract, or a mixture of recombinantly produced purified enzymes or a cell extract augmented with a recombinant purified enzyme.

The target DNA sequence is preferably a plasmid, bacteriophage, or bacterial artificial chromosome (“BAC”) that can be used to transform an expression host such as a bacteria. In a preferred embodiment, the plasmid is supercoiled. The target DNA is preferably a screenable marker such as an antibiotic resistance gene or a lacZ gene.

The oligonucleotide of the present invention is any oligonucleotide or oligonucleotide derivative that can be used to introduce a site specific, predetermined genetic change in a cell. As used herein, an oligonucleotide consists of a DNA duplex consisting of less than or equal to 200 deoxyribonucleotides and contains nucleotide derivatives such as RNA or modified sugar backbones (methylphosphonate, phosphoramidate, morpholino, peptide linkages, or containing different 2′-halo, 2′-alkyl or alkoxyalkyl sugars. Typically, an oligonucleotide of the present invention is characterized by being a duplex nucleotide, including nucleotide derivatives or non-nucleotide interstrand linkers, and having between 20 and 120 nucleotides or equivalently between 10 and 60 Watson-Crick nucleotide pairs. In a preferred embodiment, the oligonucleotide is substantially a duplex and contains a single 3′ end and 5′ end; accordingly, the strands of the duplex are covalently linked by oligonucleotide or nonoligonucleotide linkers. The targeting vectors can be single stranded or form a variety of structures based on self complementarity (single and double hairpins, cruciform, or tailed duplex). Also, a binary molecule formed by base pairing two oligonucleotides has been found active in the present invention.

The genetic readout system of the present invention is characterized as: (1) being deficient in mismatch repair; (2) being easily transformable; (3) having defined and easily screened genetic markers; and (4) being amenable to statistical numbers of organisms. A preferred embodiment of the present invention utilizes a genetic readout system based on the U.S. Pat. No. 6,004,804 and International Patent Application WO 9958723 to Kumar et al. In U.S. Pat. No. 6,004,804, Kumar et al. disclosed non-chimeric mutational vectors are effective substrates for the strand transfer and repair enzymes of eukaryotic and, specifically mammalian cells. In International Patent Application WO 9958723, Kumar et al. disclosed that two types of recombinagenic oligonucleotides, heteroduplex mutational vectors and vectors having a single segment of ribo-type nucleotides in the strand opposite the strand containing the 3′ end nucleotide and 5′ end nucleotide, unexpectedly give superior results when used with eukaryotic and specifically in mammalian strand transfer and repair enzymes. The term duplex mutational vectors (DMV) is used herein to refer to chimeric mutational vectors (CMV), heteroduplex mutational vectors (HDMV) and non-chimeric mutational vectors (NCMV), collectively. Note that a HDMV can be either chimeric or non-chimeric, however, the term CMV does not encompass HDMV.

The cell-free assay of the present invention will be more clearly understood with reference to the exemplary model systems described as follows and represented in detail in Example 1.

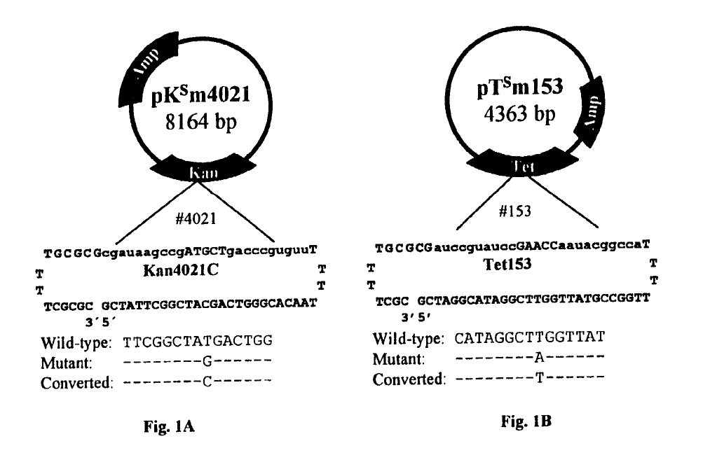

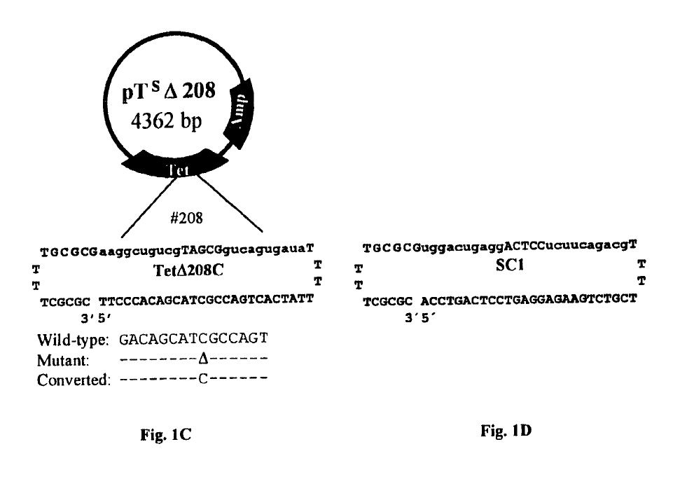

To detect gene correction, it is believed that any system known in the art which identifies the correction of point or frameshift mutations in a cell-free environment can be used. FIG. 1 indicates the plasmids used in the model systems described herein: pK^{S}m402l, pT^{S}m153, and pT^{S}Δ208. Preferably, a system using plasmid molecules containing point or frameshift mutations in the coding regions of antibiotic resistance genes is used. In the exemplary models shown herein, plasmid pK^{S}m4021 which contains the mutated kanamycin gene and a wild-type ampicillin resistance gene, plasmid pT^{S}m153 which contains a mutated tetracycline gene and a similar ampicillin resistance gene were utilized. The presence of the ampicillin gene enables control and normalization of the transfection process. In the in vitro reaction, the plasmid and appropriate chimera were mixed with the extract. After a defined time, the plasmid DNA was extracted and transformed into competent Escherichia coli cells harboring a mutation in the RecA gene. Previous results established the need for functional RecA protein in the bacterial system (Metz, et al. 1998. “Molecular mechanism of chimeric RNA/DNA oligonucleotide directed DNA sequence alteration,” Conference Proceedings: 1st Annual Meeting of the American Society of Gene Therapy, Seattle, Wash., p. 164e). Hence, the use of cells deficient in RecA function ensured that any correction observed after the phenotypic readout had occurred in the cell-free extract. These correction events were scored by selection on agar plates containing kanamycin. A dilution from the same transformation were plated in duplicate and selected on plates containing ampicillin to normalize the efficiency of electroporation. Frequencies were calculated as kanamycin revertant colonies relative to ampicillin colonies selected from the same reaction sample.

A final, but important feature of plasmid pK^{S}m4021 was the target sequence itself. Wild-type sequence conferring antibiotic resistance contains a T residue at position 4021. This base was mutated to a G, disabling functional gene activity. To avoid the possibility of positive results emanating from contaminating sources, the chimera was designed to convert the G residue to a C, instead of a T. This conservative replacement generated functional protein thereby preserving the phenotypic readout as kanamycin resistance.

FIG. 1 also displays a second substitutory system utilizing a point mutation in the gene responsible for tetracycline resistance at position 153. Similar to the kan system, a chimera, Tet153T, was designed to correct the mutation. To insure that the mutated plasmid was corrected, enabling tetracycline resistance, and avoiding artifactual results due to contamination of wild-type plasmids, a silent mutation (A [symbol] G) was engineered at position 325. DNA sequence verification of antibiotic resistant colonies was performed so that both sites, 153 and 325, were analyzed simultaneously.

FIG. 1 also illustrates the series of chimeric oligonucleotides used in this study. Kan4021C, Tet153, and TetΔ208C directed correction, while SC1 was a nonspecific control chimera which did not elicit any change.

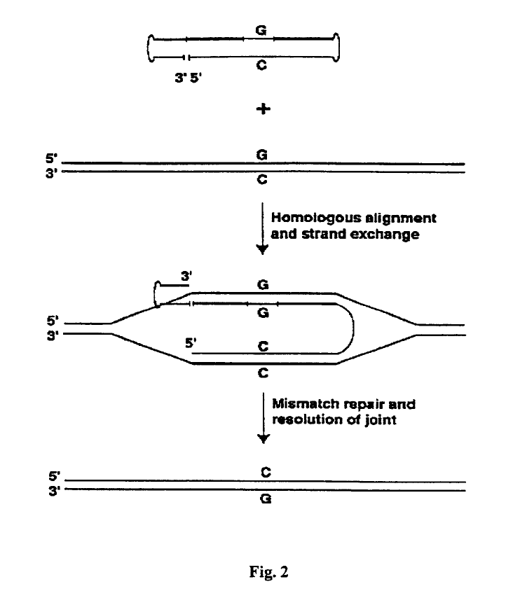

FIG. 2 outlines the mechanism of correction directed by these chimeric oligonucleotides. Upon target recognition, the chimeric oligonucleotide forms a joint molecule known as a complement-stabilized D-loop. Once stabilized, the complex is recognized by the cell's inherent DNA repair activities and through interaction with the appropriate factors, sets in motion a cascade of events that directs nucleotide exchange or insertion at the specific site on the target strand.

Extracts from monocots and dicots, as well as embryonic tissue can be used in the present invention. As illustrated in the model systems described herein, embryos were obtained from maize seeds; the source of monocot extract was Musa acuminata, while tobacco served as the source for the dicot extract. Cell-free extracts were prepared using the strategy of Cole-Strauss et al. (1999) with slight modifications as outlined in the Methods section given below. Central among the changes was the use of liquid nitrogen to freeze the samples for grinding with a mortar and pestle. The extract was prepared in 20 mM HEPES (pH 7.5), 5 mM KCI, 1.5 MM MgC1₂, 10 mM DTT, 10% glycerol and 1% PVP. The extract was mixed with plasmid DNA and the chimeric oligonucleotide in a reaction buffer containing ATP, CTP, GTP, UTP, dATP, dCTP, dGTP and dTTP. After incubation, the samples were extracted with phenol/chloroform and precipitated with ethanol. The plasmid DNA was then electroporated into a mutant strain of E. coli, containing a mutation in the RecA gene (DH10B). The bacteria were plated on agar containing the antibiotic kanamycin and allowed to grow for 18 hours at 37° C.

Table I presents the results of one study in which kanamycin resistant colonies were reacted in the presence of maize embryo and tobacco extracts. The results indicate that the buffer system used in the in vitro reaction is critical in successful gene conversion.

TABLE I
Gene Conversion of Kanamycin Resistant Mutants

| | | | Extract | | kan^{r} colonies/ |
|---|---|---|---|---|---|
| | Plasmid | Chimera | (0.05 mg/ml) | Buffer | 10⁶ amp^{r} colonies |
| 1 | + | − | − | + | 0 |
| 2 | − | + | − | + | 0 |
| 3 | + | + | − | + | 0 |
| 4 | + | + | maize embryo | − | 0 |
| 5 | + | + | maize embryo | + | 17 |
| 6 | + | + | tobacco | − | 0 |
| 7 | + | + | tobacco | + | 685 |

As seen in Table II, kanamycin resistant colonies were present in samples containing the maize embryo, Musa and tobacco extracts. There appeared to be no significant difference in the frequency of resistant colonies between monocot and dicot extracts, but a reduced number of colonies appeared when the conversion reaction occurred in the embryo extract. The number of colonies in each table reflects colony count per 10⁷ ampicillin resistant colonies. Similar results were obtained in the tetracycline system with all three extracts enabling correction. The same trend was apparent as the monocot and dicot extract contain a significantly higher level of repair activity when compared to the maize embryo extract. The conversion required for kan resistance is G [symbol] C while tetracycline resistance is conferred upon A [symbol] T transversion, and the base pair mismatches create by the respective chimeras are G/G and A/A, respectively. Both are purine-purine mismatches. The response in both systems was dose-dependent and successful correction relied on the presence of the designated extract. The maximal frequency of conversion observed in these experiments was approximately 0.08%.

TABLE II
Correction of Mutant Antibiotic Resistance Genes^{a}

Kanamycin Substitution System

| | | | | | | Kan^{r} colonies/ |
|---|---|---|---|---|---|---|
| | | | Extract (μg) | | | 10⁷ amp^{r} |
| | Plasmid | Chimera | Tobacco | Maize | Musa | colonies |
| 1 | pK^{s}m4021 | Kan4021C | 3 | — | — | 79 |
| 2 | | | 10 | — | — | 236 |
| 3 | | | 20 | — | — | 685 |
| 4 | | | — | 3 | — | 7 |
| 5 | | | — | 10 | — | 105 |
| 6 | | | — | 20 | — | 217 |
| 7 | | | — | — | 3 | 86 |
| 8 | | | — | — | 10 | 366 |
| 9 | | | — | — | 20 | 831 |
| 10 | | — | — | — | — | 0 |
| 11 | — | Kan4021C | — | — | — | 0 |
| 12 | pK^{s}m4021 | SC1 | — | — | — | 0 |

Tetracycline Substitution System

| | | | | | | Tet^{r} colonies/ |
|---|---|---|---|---|---|---|
| | | | Extract (μg) | | | 10⁷ amp^{r} |
| | Plasmid | Chimera | Tobacco | Maize | Musa | colonies |
| 1 | pT^{s}m153 | Tet153T | 3 | — | — | 16 |
| 2 | | | 10 | — | — | 114 |
| 3 | | | 20 | — | — | 285 |
| 4 | | | — | 3 | — | 3 |
| 5 | | | — | 10 | — | 17 |
| 6 | | | — | 20 | — | 83 |
| 7 | | | — | — | 3 | 27 |
| 8 | | | — | — | 10 | 139 |
| 9 | | | — | — | 20 | 364 |
| 10 | | — | — | — | — | 0 |
| 11 | — | Tet153T | — | — | — | 0 |
| 12 | pT^{s}m153 | SC1 | — | — | — | 0 |

^{a}Each reaction contained 1 μg plasmid DNA and either 1.4 μg Tet153T, 1.2 μg. Kan4021C or 1.4 μg SC1. Genetic readout utilized DH10B (recA1) cells. All experimental values reflect an average of 4 independent experiments.

A series of control experiments was performed to establish which components of the reaction were necessary for repair (Table III). Complete mixtures produced a similar number of colonies compared to the data presented in Table II, while the absence of plasmid, chimera or extract resulted in no antibiotic resistant colonies. Requirements were the same for the tetracycline system (data not shown). Important controls are represented in lines 10-12 of Table III wherein the plasmid and the chimera were incubated separately with the extract, the DNA purified and mixed prior to electroporation. With these reaction parameters, no colonies were observed, reinforcing the fact that the measured correction events occurred in the plant cell extract and not in the bacterial cells.

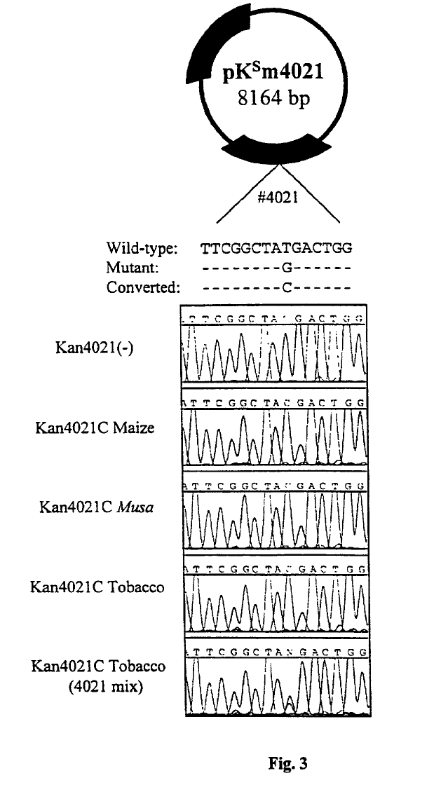

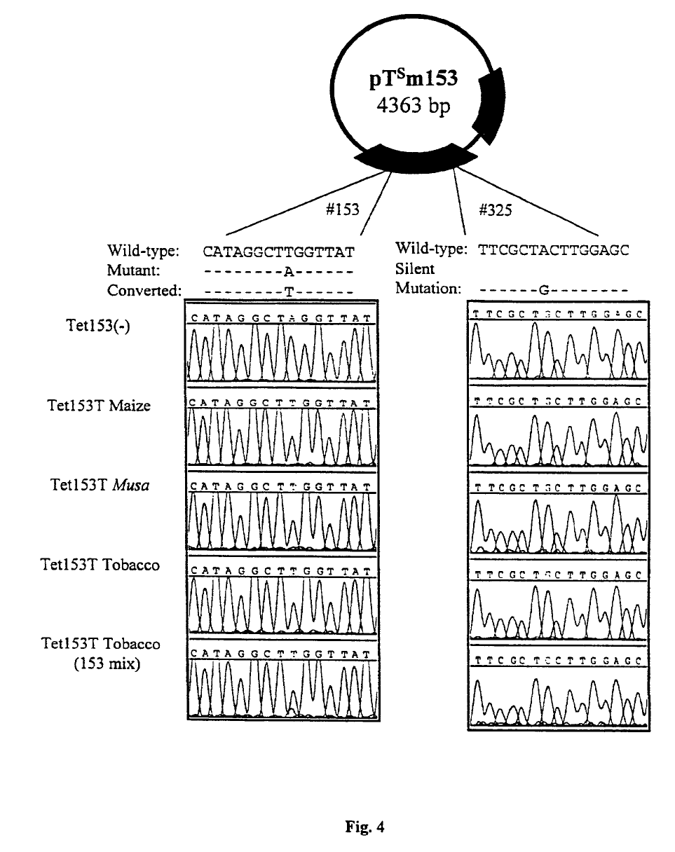

Conversion at the DNA level was measured by sequencing plasmids isolated from antibiotic resistant bacterial colonies. As seen in FIG. 3, the kanamycin sensitive mutant base G was converted to the base C. Sequencing of 20 randomly selected colonies gave the same results when either the maize embryo extract or the Musa extract catalyzed the reaction. Sequencing of the non-coding strand confirmed that both strands were repaired. Similar results were obtained in the tetracycline system (FIG. 4), wherein an A to T conversion was observed. FIG. 4 also presents the coding strand of the tetracycline gene harboring the silent mutation, and in all cases, the G residue was present. Hence, these results show that the change from antibiotic sensitivity to antibiotic resistance was the result of a unique nucleotide exchange at position 4021 (kan^{r}) or 153 (tet^{r}).

TABLE III
Chimera-directed Gene Repair Relies on
Plant Extract and Oligonucleotides^{a}

Kanamycin Substitution System

| | | | | | | Kan^{r} colonies/ |
|---|---|---|---|---|---|---|
| | | | Extract (μg) | | | 10⁷ amp^{r} |
| | Plasmid | Chimera | Tobacco | Maize | Musa | colonies |
| 1 | pK^{s}m4021 | Kan4021C | 20 | — | — | 571 |
| 2 | pK^{s}m4021 | Kan4021C | — | 20 | — | 190 |
| 3 | pK^{s}m4021 | Kan4021C | — | — | 20 | 713 |
| 4 | pK^{s}m4021 | — | 20 | — | — | 0 |
| 5 | pK^{s}m4021 | — | — | 20 | — | 0 |
| 6 | pK^{s}m4021 | — | — | — | 20 | 0 |
| 7 | — | Kan4021C | 20 | — | — | 0 |
| 8 | — | Kan4021C | — | 20 | — | 0 |
| 9 | — | Kan4021C | — | — | 20 | 0 |
| 10 | #4 + #7 | | — | — | — | 0 |
| 11 | #5 + #8 | | — | — | — | 0 |
| 12 | #6 + #9 | | — | — | — | 0 |
| 13 | pK^{s}m4021 | Kan4021C | 20^{b} | — | — | 0 |
| 14 | pK^{s}m4021 | Kan4021C | — | 20^{b} | — | 0 |
| 15 | pK^{s}m4021 | Kan4021C | — | — | 20^{b} | 0 |

^{a}Each reaction contained 1 μg plasmid DNA and 1.4 μg oligonucleotide with electroporation carried out in a DH10B E. coli strain. For #10, 11 and 12, reaction mixtures indicated were processed separately and combined prior to electroporation.
^{b}Extract boiled for 10 minutes prior to adding to the reaction mix.

The activity of the tobacco cell-free extract also produced kanamycin and tetracycline resistant colonies, but the targeted nucleotide exhibited a mixed base sequence for both genes (see FIG. 3—tobacco “4021 mix” and FIG. 4—tobacco “153 mix”). In the kanamycin system, the mixed nucleotides are C (converted) and G (mutant) (peak indicating sequence containing G has the greater surface area under the curve) evidenced by the placement of an “N” at the targeted sequence site. In the tetracycline system, the mixture is A and T, and while an “N” is not inscribed, a mixture T and A peaks is apparent (peak indicating sequence containing T has the greater surface area under the curve. Since the silent mutation (G) is present at position 325, the mixed sequence was not the result of wild-type plasmid contamination. Rather, the mixed sequence resulted from either correction of only one of the two strands, followed by plasmid replication, or from correction of only a subset of the plasmid molecule population.

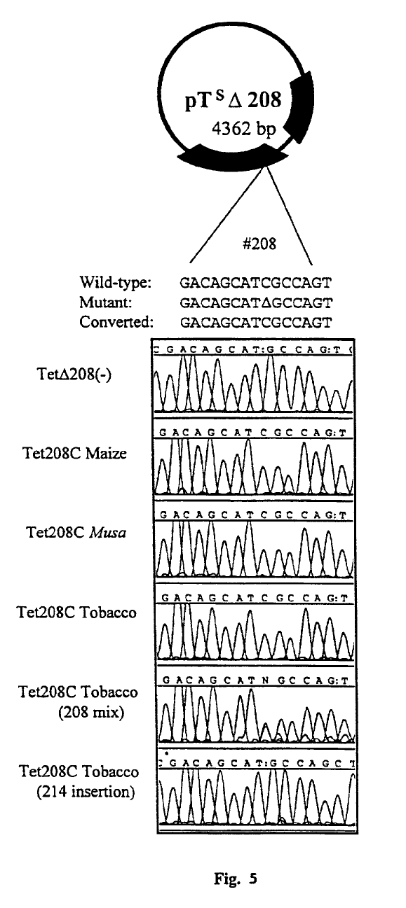

As shown above, the cell-free extracts repaired mismatched base pairs or point mutations. Using the same genetic readout system, the capacity of these extracts to repair frameshift mutations was also tested. As shown in FIG. 1, the plasmid pT^{S}Δ208 contains a deleted base at position 208 rendering the tet^{r} gene nonfunctional. Chimeric oligonucleotide TetΔ208C was designed to correct this mutation by directing the insertion of a C residue. The insertion event restores the reading frame enabling antibiotic resistance. Table IV indicates that each plant extract had the capacity to repair frameshift mutations directed by the chimera. The frequency of conversion was at least one log lower than that observed in the repair of point mutations. FIG. 5 displays the DNA sequence of mutant and converted plasmids, the latter picked from colonies growing on tet⁺ agar plates. The extracts generated from maize and Musa produced targeted gene repair at position 208 in all the clones sequenced. The results from the tobacco extract experiments were, however, different. While some clones contained the designed T insertion at the correct site, another population of tetracycline resistance colonies was found. These molecules contained either a mixture of G and A residues at the correct position or a C residue six bases downstream from the targeted site. Interestingly, this alternative insertion site was the only one found in the clones, and as in the case of the point mutation conversion, was only present in experiments using the tobacco extract.

TABLE IV
Plant Extracts Correct Single Base Deletions
Directed by Chimeric Oligonucleotides^{a}

Tetracycline Deletion System

| | | | | | | Tet^{r} colonies/ |
|---|---|---|---|---|---|---|
| | | | Extract (μg) | | | 10⁷ amp^{r} |
| | Plasmid | Chimera | Tobacco | Maize | Musa | colonies |
| 1 | pK^{s}Δ208 | TetΔ208C | 20 | — | — | 6 |
| 2 | | | 20 | — | — | 31 |
| 3 | | | — | 10 | — | 0 |
| 4 | | | — | 20 | — | 14 |
| 5 | | | — | — | 10 | 12 |
| 6 | | | — | — | 20 | 50 |
| 7 | | | — | — | — | 0 |
| 8 | — | | — | — | — | 0 |
| 9 | pT^{s}Δ208 | — | — | — | — | 0 |

aEach reaction contained 1 μg plasmid DNA and 1.4 μg oligonuclcotide. DH10B cells were used for genetic readout.

As demonstrated in the model systems wherein chimeric oligonucleotides were used for correction of point and frameshift mutations in cell-free extracts from plants, extracts from both monocot and dicot cells, as well as from embryonic tissue support the repair of these mutations. By using mutant strains of E. coli lacking RecA protein activity as a genetic readout system, the results establish sustained inheritance and clonal expansion of corrected DNA templates. Sequence analyses of these clones confirm genetic repair at the DNA level.

The cell-free assay system of the present invention offers several advantages over eukaryotice cell-based methods known in the art. One significant advantage is apparent in the area of designing new chimeric oligonucleotides for a given plant tissue, which include variations in the RNA length, mismatches within the chimera itself, the placement of the nick or open phosphodiester bond, and modifications of the phosphodiester backbone, wherein the most recombinagenic chimera for testing in a given plant tissue is identified and the type of chimera which produces the correct gene change versus those that produce aberrant changes is determined. Current methods which use intact plant tissue demonstrate gene conversion using chimera are difficult and time-consuming. The assay system of the present invention, however, provides a timely and precise way to measure the effectiveness of new chimera designs in a biochemically defined reaction.

The assay of the present invention can be used to determine whether there is a difference in the rate of successful targeting if the cells are in a particular cell cycle phase or whether they are mitotic or meiotic. Cell-free extracts can be produced from various staged cells or those that are meiotic in an effort to define the most optimal time for targeting. The precision of the targeted event can also be ascertained using this system, for example, is the correct or incorrect base more likely to be targeted in meiotic cells. Again, the cell-free assay offers an advantage over difficult, time-consuming tissue experiments using tissue such as germ tissues or embryos.

In gene conversion experiments, random mutagenesis can make it difficult to assess a particular trait of interest if a random mutagenic event alters the expected phenotypic change caused by treatment with a chimeric oligonucleotide. With the cell-free assay of the present invention, the rate of random mutagenesis can easily be determined. By determining the ratio of gene correction to random mutagenesis (Correction/Mutagenesis or C/M ratio), the selection of optimum target plant tissue and chimeric oligonucleotide for studying a given gene conversion is made possible.

The assay of the present invention can be used to readily assess whether a given plant or plant tissue has sufficient enzymatic machinery to catalyze the reactions necessary for gene conversion. If the proteins that are likely to be involved in chimera-directed activity are absent or in low abundance, then the chances of seeing a phenotypic change after treatment are greatly reduced. Such information would be helpful in selecting tissue or plant type to target.

The assay of the present invention can also be used to demonstrate what types of DNA repair proteins are present in plant cell(s) from a selected plant tissue. This assay system provides a means by which such proteins and eventually their genes can be isolated. The cell-free extract can be fractionated, and biochemical purification of the active proteins can be enabled. For any purification protocol, the single most important aspect is a reliable assay system to follow the activity. The cell-free extract provides such a test system.

The assay system of the present invention can be used to determine the optimal cell culture conditions to increase efficiency of chimera-mediated gene conversion in a biochemically defined environment. While the effect of plant or plant cell growth conditions (including medium components such as growth regulators or hormones) can have an effect on the chimera-directed gene conversion efficiency, the assay of the present invention provides a means for determining how modification(s) of growth conditions can alter chimera efficiency.

The assay system of the present invention provides a means by which gene conversion mechanisms can be elucidated. Preliminary evidence suggest that fundamental differences may exist between the types of gene conversions observed in monocot and dicot plant species. Using the assay of the present invention, these differences in gene conversion can be studied and the molecular components corresponding with these differences elucidated in a biochemically defined reaction.

The assay of the present invention can be used to determine if environmental stimuli increase the efficiency of chimeras in plant cells, i.e., if exposure of plants or plant cells to chemical mutagens, UV, gamma, or other high energy sources stimulate cellular machinery resulting in a corresponding increase in chimera efficiency. Likewise, the molecular components associated with the response to environmental stimuli can be identified.

The assay of the present invention can be used to characterize mutant plant lines. The extract system will be a testing ground for determining the molecular basis for certain plant mutants such as those that are, but not limited to, UV sensitive and deficient in DNA repair. It can also be used to identify proteins that suppress or enhance gene targeting efficacy for which agonists and antagonists can be designed, e.g., small molecule, peptide, antisense RNA, interference RNA (RNAi), or ribozyme.

### EXAMPLE 1

### Cell Free Assay

The cell-free assay of the present invention was performed using plasmids having kanamycin and tetracycline selectable markers, chimeric RNA/DNA oligonucleotides, and cell-free extracts were prepared from imbibed maize embryos and tobacco Nt-I and Musa cell.

Plasmids

Kanamycin and tetracycline selectable markers were used in two substitutory systems to determine nucleotide exchange in the cell-free extracts. The kanamycin sensitive plasmid pK^{S}m4021 contains a single base transversion (T [symbol] G), which creates a TAG stop codon in the kanamycin (kan) gene at codon 22. A tetracycline sensitive plasmid pT^{S}m153 carries a single T [symbol] A nucleotide change at position 153 in the pBR322 plasmid, which creates a stop codon in the tetracycline (tet) gene at codon 23. A nucleotide insertional system with a tetracycline sensitive plasmid, pT^{S}Δ208, was used to analyze repair of single base deletions in cell-free extracts. The plasmid carries a single nucleotide deletion at position 208, which creates a frameshift in the tet gene of pBR322 at codon 41. The plasmids also contain a wild-type ampicillin gene used for propagation and normalization (Cole-Strauss et al., 1999).

Oligonucleotides

Synthetic oligonucleotides were used to direct reversion of kan^{S} and tet^{S} genes to restore resistance to their respective antibiotics. Chimeric RNA/DNA oligonucleotides, Kan4021C and KanGGrv, which can direct conversion of the kan^{S} gene in pK^{S}m4021 at codon 22 from TAG to TAC (stop tyrosine), were synthesized as previously described (Cole-Strauss et al., 1999). Chimeric RNA/DNA oligonucleotides Tet153C and TetΔ208C were used to revert the tets genes of plasmids pT^{S}m153 and pT^{S}Δ208, respectively at their mutated bases. A non-specific chimera SC1 (Cole-Strauss et al., 1996) was used for comparison and as a control.

Plant Materials

Maize seeds (Pioneer Hybrid line B3733) were imbibed in a sealable plastic box containing paper towels soaked in distilled water. Seeds were incubated at 25° C. for 18 to 36 hours. Embryos were dissected from imbibed seed at 18, 24, and 36 hours post-imbibition, frozen in liquid nitrogen and stored at −80° C. Tobacco Nt-1 cell suspensions were maintained as shaker cultures (27° C., 200 rpm in a 250 ml flask) and transferred weekly to fresh suspension medium (CSM) containing: Murashige and Skoog salts (Gibco BRL, Grand Island, N.Y.), 500 mg/l 2-(4-morpholino)ethanesulfonic acid (MES), 1 mg/l thiamine, 100 mg/l myoinositol, 180 mg/1 KH₂P04, 2.21 mg/l 2,4 dichlorophenoxyacetic acid (2,4-D), and 30 g/l sucrose (pH 5.7). Musa acuminaia (banana) cv Rasthali cell suspensions (the kind gift of T. R. Ganapathi) were maintained as shaker cultures (27° C., 80 rpm in a 125 ml flask) and transferred every 10 days to fresh M2 cell suspension medium (Cote, et al. 1996. “Embryogenic cell suspensions from the male flower of Musa AAA cv. Grand Nain,” Physiol Plant 97:285-290). Dense Nt-1 and Musa cell suspensions were centrifuged in 50 ml disposable centrifuge tubes at 700× g for five minutes at room temperature. Following centrifugation, the liquid medium was decanted, and the pelleted cells were frozen in liquid nitrogen and stored at −80° C.

Preparation of Cell-Free Extracts

Cell-free extracts were prepared from imbibed maize embryos and tobacco Nt-1 and Musa cell suspensions by a modification of Cole-Strauss et al. (1999). Plant samples were ground under liquid nitrogen with a mortar and pestle. Then, 3 ml of the ground plant tissue were extracted in 1.5 ml of extraction buffer (20 mM N-[2-hydroxyethyl]piperazine-N′-[2-ethanesulfonic acid] (HEPES), pH 7.5, 5 mM KCl, 1.5 mM MgC1₂, 10 mM dithiothreitol (DTT), 10% glycerol, and 1% polyvinylpyrrolidone (PVP). Samples were then homogenized with 15 strokes of a Dounce homogenizer. Following homogenization, samples were incubated on ice for one hour and centrifuged at 3000× g for five minutes to remove plant cell debris. Protein concentrations of the supernatants were determined by Bradford assay (Bradford, M. 1976. Anal Biochem 72:248). Extracts were dispensed into 100 μg aliquots, frozen in a dry ice-ethanol bath and stored at −80° C.

In Vitro Reaction

One reaction mixture consisted of 1 μg of substrate plasmid pK^{S}m4021 and 1.4 μg of either effector chimeric oligonucleotide Kan4021C or KanGGrv for the kan^{S} system. For the tet^{S} system, the reaction mixtures consisted of 1 μg of substrate plasmid pT^{S}m153 and 1.4 μg of effector oligonucleotide Tet153C, or pT^{S}Δ208 and 1.4 μg of effector oligonucleotide TetΔ208C. These components were mixed in a buffer of 20 mM Tris 7.6; 15 mM MgCl₂; 1 mM DTT; 0.2 mM spermidine; 2.5 mM ATP; 0.1 mM each of CTP, GTP, and UTP; 0.01 mM each of dATP, dCTP, dGTP, and dTTP; 0.1 mM NAD; and 10 μg/ml bovine serum albumin (BSA). The reaction was initialized by adding plant cell-free extracts to 0.1 to 0.8 mg/ml in 100 μl volumes. The reactions were incubated at 30° C. for 1 hour and stopped by placing on ice. The substrate plasmid was then isolated by phase partition with phenol, one chloroform extraction, followed by ethanol precipitation on dry ice for 1 hour and centrifugation at 4° C. for 30 minutes.

Electroporation, Plating and Selection

Five μl of resuspended reaction precipitates were used to transform 20 μl aliquots of electrocompetent DH10B bacteria using a Cell-Porator apparatus (Life Technologies, Rockville, Md.) as described by the manufacturer. Each mixture was transferred to a 1 ml SOC (per liter: 10 g Bacto-tryptone, 5 g Bacto yeast extract, 5 g NaCl, 10 mM KCl, 10 mM MgCl₂, and 10 mM glucose at pH 7.0) culture and incubated at 37° C. for 1 hour. Then, converted plasmids were amplified by adding kanamycin to 50 μg/ml or tetracycline to 12 μg/ml and an additional incubation for 3 hours at 37° C. Next, 100 μl aliquots of undiluted cultures were plated onto LB agar plates containing 50 μg/ml kanamycin or 12 μg/ml tetracycline, respectively. Also, 100 μl aliquots of a 10⁴ dilution of the cultures were plated onto LB agar plates containing 100 μg/ml ampicillin. Plating was performed in duplicate using sterile Pyrex beads. Both sets of plates were incubated for 16-18 hours at 37° C., and colonies were counted using an Accucount 1000 plate reader (Biologics, Inc., Gainesville, Va.). Targeted conversion of the kan^{S} gene was determined by normalizing the number of kanamycin resistant colonies by dividing by the number of ampicillin resistant colonies, since all plasmids contain a wild type amp gene. Similarly, targeted conversion of the tet^{S} gene was determined by normalizing the number of tetracycline resistant colonies by dividing by the number of ampicillin resistant colonies. Resistant colonies were confirmed by selecting isolated clones for mini preparation of plasmid DNA followed by sequencing using an ABI Prism kit on an automated ABI 310 capillary sequencer.

We claim:

1. A method of modifying a target site of a gene-of-interest comprising:
a. providing an oligonucleotide that encodes a modification of a gene-of interest,
b. providing a duplex DNA molecule containing said gene-of-interest operably linked to a promoter so that said gene of interest can be expressed in a host organism,
c. providing a cell-free enzyme mixture comprising recombination and gene repair activities and a mismatch repair activity;
d. reacting said oligonucleotide, said duplex DNA molecule, and said cell-free enzyme mixture whereby said gene-of-interest is modified at said target site to form a modified gene of interest;
e. introducing said modified gene-of-interest into said host organism; and
f. detecting the expression of said modified gene-of-interest.

2. The method of claim 1, wherein said oligonucleotide comprises at least 20 and less than or equal to 200 nucleotides.

3. The method of claim 1, wherein said oligonucleotide comprises at least 10 and less than or equal to 100 Watson-Crick nucleotide pairs.

4. The method of claim 1, wherein said oligonucleotide comprises a single 3′ end and a single 5′ end.

5. The method of claim 1, 2, 3 or 4, wherein said expression of said modified gene-of-interest confers a selectable trait on said organism.

6. The method of claim 1, 2, 3 or 4, wherein said expression of said modified gene-of-interest confers an observable trait on said organism.

7. A method of altering a DNA sequence comprising:
a. providing an oligonucleotide that encodes a modification of a DNA sequence,
b. providing a duplex DNA molecule containing said DNA sequence,
c. providing a cell-free enzyme mixture comprising recombination and gene repair activities and a mismatch repair activity,
d. reacting said oligonucleotide, said duplex DNA molecule and said cell-free enzyme mixture comprising said recombination and gene repair activities and a mismatch repair activity, whereby said DNA sequence is modified to form an altered DNA sequence, and
e. detecting said altered DNA sequence.

8. The method of claim 7, further comprising fractionating a cell-free composition so as to enrich said altered DNA sequence relative to said DNA sequence, prior to detecting said altered DNA sequence.

9. The method of claim 7 or 8, wherein said oligonucleotide comprises at least 20 and less than or equal to 200 nucleotides.

10. The method of claim 7 or 8, wherein said oligonucleotide comprises at least 10 and less than or equal to 100 Watson-Crick nucleotide pairs.

11. The method of claim 7 or 8, wherein said oligonucleotide comprises a single 3′ end and a single 5′ end.

12. The method of claim 7 or 8, wherein said oligonucleotide is a duplex mutational vector comprising a contiguous single-stranded self-complementary oligonucleotide having a 3′ end and a 5′ end, wherein said 3′ end and said 5′ end are juxtaposed and wherein at least five contiguous nucleotides are Watson-Crick base paired, the sequence of said oligonucleotide comprising a template for said modified DNA sequence.

13. A cell-free composition for the modification of a DNA sequence comprising:
a. a duplex DNA containing a target sequence;
b. an oligonucleotide which targets the DNA sequence and encodes the modification thereof;
c. a cell-free enzyme mixture comprising recombination and gene repair activities; and
d. a reaction buffer.

14. The composition of claim 13, wherein said oligonucleotide comprises at least 20 and less than or equal to 200 nucleotides.

15. The composition of claim 13, wherein said oligonucleotide comprises at least 10 and less than or equal to 100 Watson-Crick nucleotide pairs.

16. The composition of claim 13, wherein said oligonucleotide comprises a single 3′ and a single 5′ end.

17. The composition of claim 13, wherein said duplex DNA sequence is a portion of a gene-of-interest that is operably linked to a promoter, so that said gene-of-interest can be expressed in a host organism.

18. The composition of claim 13, wherein said cell-free enzyme mixture lacks mismatch repair activity.

19. The composition of claim 18, wherein said recombination and gene repair activities are provided by a eukaryote-derived enzyme.

20. The composition of claim 19, wherein said cell-free enzyme mixture is a defined enzyme mixture of purified plant, yeast or mammalian recombination and repair proteins capable of catalyzing gene repair.

21. The composition of claim 19, wherein said cell-free enzyme mixture is an extract of a eukaryotic cell.

22. The composition of claim 21, wherein said cell-free enzyme mixture is an extract of a plant cell.

23. The composition of claim 13, wherein said cell-free enzyme mixture further comprises a mismatch repair activity.

24. The composition of claim 23, wherein said mismatch repair activity is provided by a eukaryote-derived enzyme.

25. The composition of claim 24, wherein said cell-free enzyme is an extract of a eukaryotic cell.

26. The composition of claim 25, wherein said cell-free enzyme is an extract of a plant cell.

27. The composition of claim 23, wherein said recombination and gene repair activities are provided by a eukaryote-derived enzyme.

28. The composition of claim 27, wherein said cell-free enzyme mixture is a eukaryotic cell extract.

29. The composition of claim 13, wherein said oligonucleotide is a duplex mutational vector comprising a contiguous single-stranded self-complementary oligonucleotide having a 3′ end and a 5′ end, wherein said 3′ end and said 5′ end are juxtaposed and wherein at least five contiguous nucleotides are Watson-Crick base paired, the sequence of said oligonucleotide comprising a template for said modified DNA sequence.
